(19) United States
(12) Patent Application Publication
(10) Pub. No.: US 2006/0173390 A1
(43) Pub. Date: Aug. 3, 2006

(54) Immobilizing assembly and methods for use in diagnostic and therapeutic procedures

(76) Inventors: Robert Van Wyk, Largo, FL (US); Berndt Schmit, Seattle, WA (US); Mark Keeton, Salt Lake City, UT (US); Ben Babusis, Sammamish, WA (US)

Correspondence Address: SMITH PATENT CONSULTING CONSULTING, LLC, P.O. BOX 2726, ALEXANDRIA, VA, 22301, US

(21) Appl. No.: 11/342,574

(22) Filed: Jan. 31, 2006

Related U.S. Application Data
(60) Provisional application No. 60/648,590, filed on Jan. 31, 2005.

(51) Int. Cl. A61F 5/00

(52) U.S. Cl. 602/6; 602/5

(57) ABSTRACT

Herein described is an apparatus and method for stabilizing, restraining and positioning a patient during human medical or veterinary procedures, for example, diagnostic imaging procedures, such as Magnetic Resonance Imaging (MRI) and Computerized Tomography scanning procedures, or therapeutic procedures, such as stereotactic radiosurgery. The restraining apparatus of the present invention, comprised of a castable sleeve and optional expandable element, sufficiently immobilizes the patient so as to eliminate motion artifact and motion degradation, to thereby provide improved imaging and/or therapy results.

### PRIORITY

This application claims the benefit of U.S. Provisional Application Serial No. 60/648,590, filed Jan. 31, 2005.

### TECHNICAL FIELD OF THE INVENTION

The present invention relates generally to stabilizing, restraining, and positioning a portion of the body of a subject during human medical and veterinary procedures. More specifically, the present invention relates to an assembly and method for immobilizing yet comfortably positioning a subject's body, while improving image quality during Magnetic Resonance Imaging and Computerized Tomography scanning procedures, or other imaging/diagnostic or therapeutic procedures, such as radiation therapy or Gamma Knife non-invasive surgery.

### BACKGROUND OF THE INVENTION

Computerized Tomography (“CT”) scanning and Magnetic Resonance Imaging (“MRI”) are procedures used for obtaining unique cross sectional views of a subject's internal anatomy, thereby aiding in diagnosis and treatment. CT scanning involves the application of many low dosage x-rays through the body at different angles to produce cross sectional images of body tissue with the aid of a computer. MRI involves the use of electromagnets and the application of short bursts of radio waves while in a powerful magnetic field, rather than x-rays, through the body. The bursts stimulate the hydrogen atoms in the subject's tissue to produce a signal that a magnetic coil detects and a computer transforms into an image.

Both of these procedures require absolute immobility in the area of the body being imaged. Subject motion is an ever-present problem for the radiologist. During the actual sequence, the subject must remain absolutely motionless or the images will be blurred. Subject movement often renders the images uninterpretable and/or compromises the accuracy of the exam, which, in turn, can potentially harm the subject. This disruption in the images is known as “motion artifact.”

Motion artifact is a constant problem in all MRI because this procedure requires a relatively long period of time to obtain the images. In MRI, the subject must remain motionless for multiple imaging sequences that comprise the total exam. The exam may last 30 to 60 minutes and each sequence typically takes about 2 to 9 minutes to run. While CT scanning involves much shorter imaging times than MRI, there are motion considerations in subjects who are unable to cooperate. Many head CT scans are performed on acutely injured patients and those with sudden changes in mental status. Both groups of subjects are compromised in their ability to take instruction and/or remain still and, therefore, would benefit from a motion-limiting or motion-restricting device.

In both MRI and CT scans, maintaining absolute stillness can be a challenge for an otherwise healthy adult. For one afflicted with tremors (such as in Parkinson's Disease), pediatric subjects, subjects with altered mental status from stroke or trauma, intoxicated subjects, and those subjects who simply fall asleep during the imaging test and are twitchy sleepers, maintaining the requisite immobility can be virtually impossible. Similar issues arise when scanning animal subjects, even when sedated.

Subject motion can be divided into two categories: macromotion and micromotion. Macromotion occurs on the scale of centimeters and results in the body part of interest actually moving, partly or completely, out of the field of view. This results in images that do not fully include the body part of interest. The subject then has to be “re-scouted” to locate the position of the body part and the sequence repeated once the body part has been re-localized. This results in a loss of several minutes. Micromotion occurs on a scale of millimeters and may be the result of a patient tremor, cardiac pulsation, breathing, subject restlessness, or subject discomfort resulting in unconscious twitching and shifting. Thus, micromotion results in blurred images, which also have to be repeated. Fortunately, the subject does not need to be re-localized for these repeat sequences.

Radiologists expend extensive effort to combat subject movement. The current practice for combating subject movement involves the use of make-shift restraints, from foam pads, pillows, and/or towels. Subjects are brought into the MRI machine (or CT scanner) and positioned with their limb or head in the appropriate coil or imaging device. The foam pads, pillows and/or towels are then used with tape and straps to stabilize the body part and obtain a comfortable position. This positioning often takes several minutes and is fraught with poor success. Subject motion occurs because the pads, pillows, etc., do not create a custom fit and are limited in their restraining ability. Likewise, the lack of custom fit cannot create or maintain subject comfort. There are inevitable pressure points that result from a fold in the pillow, a corner or seam of a pad, and/or an edge of the coil or imaging device. The subject may have started the exam feeling quite comfortable, but after a few minutes, an intolerable pressure point develops and the patient is ultimately compelled to shift his or her body. This even occurs in the normally conscious and cooperative subject, despite his best efforts to hold still. In sum, the foam pad/pillow system is neither comfortable nor does it provide an adequate level of restraint. Moreover, foam pads and pillows inherently lack the custom fit or restraint of the limb necessary to prevent all micro- and macromotion.

Motion degradation leads to a significant number of non-diagnostic studies and also to considerable waste of resources. Accordingly, efforts have been made to immobilize subjects and subject extremities for MRI through the use of various devices. For example, Marandos (U.S. Pat. No. 5,400,787) discloses an inflatable MRI sensing coil assembly positioning and retaining device. The Marandos device uses a first inflatable sleeve, disposed radially inward of an imaging coil formed of concentric rigid sleeves with foam material disposed between the rigid sleeves, to properly position the coil about the limb of a patient, and a second inflatable sleeve, disposed radially about the imaging coil, to properly position the coil and limb in the field of an MRI machine. However, while the inflatable sleeve internal to the coil, which locates and restrains the patient's limb relative to the coil, can decrease relative motion, due to its inherent flexibility, it cannot totally prevent it. This is particularly true for pivoting of the limb about the center of the coil assembly. Thus, while the Marandos device can decrease relative motion between the patient's “target section” and an MRI coil, motion artifacts will remain, particularly when using modern MRI equipment with its higher resolution.

Filler et al, in U.S. Pat. Nos. 5,560,360 and 5,706,813, disclose a system for generating diagnostically useful images of neural tissue using MRI. Therein, Filler et al. specifically describe a “splint” for reducing motion artifacts, for providing a reference frame, and for reduction of edge effects. The splint is comprised of a rigid frame and a non-rigid system”, more particularly a sleeve made of a thin film plastic and filled with a conformable substance, preferably a fluid, such as water containing gel, silicone, foam, or cobalt-chloride doped water. Fluid introduced into the sleeve from a reservoir under pressure forces the sleeve against the patient's skin, thereby immobilizing the region under examination. However, because the substance within the sleeve is conformable and “non-rigid”, relative motion between the limb and the rigid frame is reduced but not eliminated. Thus, as with the Marandos device, motion artifacts may be reduced but are not eliminated, particularly when using modern MRI equipment with its higher resolution.

Finally, Schmit et al., in U.S. Pat. Nos. 6,684,096 and 6,882,878, disclose a restraining apparatus and method for use in imaging procedures. The disclosed restraining apparatus includes castable and expandable sleeves used to fix the patient into a coil. The castable sleeve encircles the limb of a patient, and is filled with a quickly casting material. The expandable sleeve encircles the castable sleeve and is inflatable such that the expandable sleeve conforms to the inner dimensions of a particular MRI coil, CT scanner, or other imaging device. However, like Marandos and Filler, the Schmit assembly fails to include a rigid locating means between the patient's limb and the MRI coil. Accordingly, patient motions are decreased but not eliminated and, thus, some motion artifacts remain.

In sum, while the currently available restraining devices can decrease subject movement relative to an imaging coil of an MRI, due to the absence of rigid mounting means disposed between the limb and the coil, they are unable to eliminate this movement. In addition, the Marandos, Filler and Schmit devices all have sleeves formed in a manner which requires that the patient limb be inserted axially into the apparatus. Such insertion can be problematic for injured subjects or for those with altered mental status.

Furthermore, as noted above, immobilization may also be required for certain therapeutic procedures, for example, stereotactic radiosurgery. In such instances, both macro- and micromotion can substantially compromise the treatment results. Accordingly, there is a clear need in the art for new and improved immobilization techniques and devices for use during diagnostic and therapeutic procedures.

### SUMMARY OF THE INVENTION

In view of the foregoing, it is a primary object of the present invention to provide an assembly and method for comfortably positioning yet firmly restraining a subject within an MRI or CT scanner or other imaging device (hereinafter “MRI”) or radiation therapy machine, so as to immobilize the subject and improve image quality.

It is a further object of the present invention to provide an assembly and method for providing a custom fit of a subject's head, limb, or other body part within an MRI or radiation therapy machine.

It is another object of the present invention to provide an assembly and method for providing optimal placement of a subject's head, limb, or other body patent within an MRI or radiation therapy machine.

Still another object of the present invention is to provide an assembly and method having a level of restraint that substantially restricts all micro- and macro-motion of a subject's head, limb, or other body patent within an MRI or radiation therapy machine.

It is additionally an object of the present invention to provide an assembly and method for rigidly locating a subject's head, limb, or other body patent within an MRI or radiation therapy machine.

Yet another object of the present invention is to provide an assembly and method a low cost, disposable restraining device, which will decrease the time to set up a subject for scanning and/or therapy, thereby further improving productivity.

Still another object of the present invention is to provide an assembly and method that allow for enhanced subject comfort, thereby improving tolerance of the diagnostic or therapeutic procedure.

These and other objects are accomplished in the invention disclosed herein, which includes a means and method for positioning and restraining (i.e., immobilizing) a subject's head, limb, or other body part for MRI or CT examination, or for radiation therapy or Gamma Knife non-invasive surgery, by rigidly locating the subject within the machine. The subject is restrained in a rigid cast formed from a quick-set polymeric foam material. The inner (proximal) portion of the cast closely conforms to the body of the subject. The outer (distal) portion of the cast is formed to a shape having locating features for rigidly mounting within an imaging coil. In a preferred embodiment, the locating features are formed in the lower portion of the outer portion of the cast such that when the cast is placed in an imaging coil, alignment is maintained between the axis of the coil and that of the body. In this preferred embodiment, an expandable member is placed on top of the cast, in the space between the top of the cast and the upper inner (proximal) surface of the coil. In a preferred embodiment, the expandable member is an inflatable vessel. When the vessel is expanded, it exerts a downward force on the cast. This downward force is sufficient to maintain contact between the alignment features on the lower portion of the cast and the inner, lower surface of the coil. Frictional forces between the locating features of the cast and the coil inner surface are sufficient to prevent axial or rotational movement of the cast within the coil, thereby providing a rigid mounting of the cast in the coil. In this manner, the subject is located and immobilized within the coil. In another embodiment, a mechanical expanding device, placed in the gap between the top of the cast and the coil inner surface, provides the downward force rather than an expandable vessel. In yet another embodiment, a resilient member, placed on top of the cast prior to affixing of the upper coil portion to the lower portion, provides the downward force.

In other embodiments, the relative positions of the expandable member and cast may be altered. For example, the relative positions of the expandable member and cast may be reversed, whereby the expandable member is disposed underneath the cast, applying an upward force. Likewise, the expandable member may be disposed one side or the other of the cast, applying a sideways force. The criticality lies in the unidirectional nature of the force, more particularly the application of the force perpendicular to the one or more locating features of the cast.

In other embodiments for use with coils having a removable upper portion, the expandable member may be eliminated. The cast may then be formed with a plurality of protuberances on its upper surfaces, the protuberances being sufficiently sized so as to be deformed (crushed) by the upper portion of the coil when mounted to the lower portion. The deformation of the protuberances causes a downward force on the cast which, in turn, prevents movement of the cast within the coil.

In some cases, it may be advantageous to cast the subject's body directly in the coil of the MRI rather than in a mold, for example, to minimize movement and manipulation of an injured extremity. In one such cast-in-place embodiment, intended for such use, the body part, such as a limb or extremity, is positioned in the coil and the void between the coil interior space and the limb is filled with a quick-set foam casting material. When coils having a removable upper portion are used, the expandable member may be eliminated. The cast fills the entire interior of the coil. The compressive force from the expanding foam causes frictional forces which prevent movement of the cast within the coil. The cast may then be removed from the coil by removing the upper portion of the coil and lifting the cast from the coil. In a second cast-in-place embodiment, intended for use with one piece coils, an expandable member is placed on top of the cast and the expandable member is expanded before the casting material is injected into the cast, such that when the examination is complete, collapsing the collapsible member provides clearance which allows the cast to be removed axially from the coil.

The invention taught herein may also be used for immobilizing the head of a subject. In such embodiments, the casting sleeve is formed so as to surround the various portions of the head while maintaining patient comfort and breathing. The cast formed has locating features formed on its outer surface for locating in the coil. In an alternate embodiment, the cast surrounding the head upper portion may be formed in place, for example, in the MRI coil.

Because the cast conforms closely to the subject, there is an absence of pressure points which can cause subject discomfort. This, in turn, improves subject comfort during the MRI or CT exam, and minimizes subject movement associated with discomfort. Because the body part is held motionless during the exam, motion artifacts are eliminated. It is not necessary to repeat scans due to subject movement during the scans. Because all scans are usable and no scans need to be repeated, the throughput of the MRI unit is increased, which, in turn, leads to improved profitability and more efficient scheduling.

The immobilizing assembly of the present invention finds additional utility as an emergency splint, serving as a quick and effective splint for limbs injured in the field and providing safe and secure support for the injured (e.g., broken) limb during transport from the site of injury, for example in an ambulance, to the treatment site (e.g., hospital emergency room, triage center, etc.).

These and other objects and features of the invention will become more fully apparent when the following detailed description is read in conjunction with the accompanying figures and examples.

### BRIEF DESCRIPTION OF THE DRAWINGS

FIG. 1 shows an immobilizing device and method in use locating a subject's limb or extremity within the coil of an MRI.

FIG. 2 is a plan view of a castable sleeve assembly formed in accordance with the principles of this invention.

FIG. 3 is a front elevational view of the objects of FIG. 2.

FIG. 4 is an end elevational view of the objects of FIG. 2.

FIG. 5 is a perspective view of the objects of FIG. 2.

FIG. 6 is an expanded sectional view in direction A-A of FIG. 2.

FIG. 7 is a perspective view of the lower subassembly of a mold used to form the external shape of the cast of FIG. 1.

FIG. 8 is a perspective view of the upper subassembly of a mold used to form the external shape of the cast of FIG. 1.

FIG. 9 is a plan view of a lamination used to form the lower subassembly of FIG. 7.

FIG. 10 is a plan view of a lamination used to form the upper subassembly of FIG. 7.

FIG. 11 is a side elevational view of a mold used to form the external shape of the cast of FIG. 1.

FIG. 12 is a perspective view of the objects of FIG. 11.

FIG. 13 is an axial elevational view of the objects of FIG. 11.

FIG. 14 is a perspective view of a limb prepared for casting, wrapped in the sleeve assembly of FIG. 2, and positioned in the lower mold subassembly of FIG. 7.

FIG. 15 is a perspective view of the limb of FIG. 14, positioned within the mold of FIG. 11.

FIG. 16 is a perspective view of a limb of FIG. 14 in a cast formed in accordance with the principles of this invention, prior to removal from the mold of FIG. 11.

FIG. 17 is a perspective view of the cast limb of FIG. 16 prior to removal from the mold lower subassembly.

FIG. 18 is a perspective view of the cast limb of FIG. 17 with the casting process complete.

FIG. 19 is an axial sectional view of the coil, cast, limb, and inflatable vessel of FIG. 1.

FIG. 20 is a plan view of an expandable device of an alternate embodiment, with the expandable member retracted.

FIG. 21 is a side elevational view of the objects of FIG. 20.

FIG. 22 is a perspective view of the objects of FIG. 20.

FIG. 23 is an end view of the objects of FIG. 20.

FIG. 24 is an axial sectional view of the objects of FIG. 20 at location A-A.

FIG. 25 is a plan view of an expandable device of an alternate embodiment, with the expandable member extended.

FIG. 26 is a side elevational view of the objects of FIG. 25.

FIG. 27 is a perspective view of the objects of FIG. 25.

FIG. 28 is an end view of the objects of FIG. 25.

FIG. 29 is an axial sectional view of the objects of FIG. 25 at location B-B.

FIG. 30 is a perspective view of a cast portion of an alternate embodiment.

FIG. 31 is an axial sectional view of the object of FIG. 30.

FIG. 32 is an axial sectional view of the cast portion of FIG. 30 mounted in a two-piece coil during use.

FIG. 33 is a perspective view of another alternate embodiment during use.

FIG. 34 is a perspective view of another alternate embodiment during use.

FIG. 35 is an axial sectional view through the mid-portion of the coil of FIG. 34 of the objects of FIG. 34.

FIG. 37 is a side elevational view of an alternate embodiment for immobilizing a head during use.

FIG. 37 is an axial sectional view of the objects of FIG. 31 at location A-A of FIG. 31.

### DETAILED DESCRIPTION OF THE PREFERRED EMBODIMENTS

In the context of the present invention, the following definitions apply:

The words “a”, “an” and “the” as used herein mean “at least one” unless otherwise specifically indicated.

The term “proximal” refers to that end or portion which is situated closest to the body of the subject when the device is in use.

The term “distal” refers to that end or portion situated farthest away from the body of the subject when the device is in use.

As noted previously, the instant invention has both human medical and veterinary applications. Accordingly, the terms “subject” and “patient” are used interchangeably herein to refer to the person or animal being treated or examined. Exemplary animals include house pets, farm animals, and zoo animals. In a preferred embodiment, the subject is a mammal.

As noted previously, the instant invention has both diagnostic and therapeutic utility. Accordingly, although the detailed description below often makes specific reference to use in combination with MRI or CT scanning, the present invention is equally applicable to therapeutic procedures, for example, in combination with radiation therapy machines such as the Gamma-Knife.

The accompanying figures, described in detail below, illustrate aspects of the invention but are in no way intended to limit the scope of the present invention.

The cast of the instant invention is formed in a sleeve having a proximal inner wall and a distal outer wall having an enclosed space therebetween into which an expandable casting material is injected. In a preferred embodiment, the expandable casting material is a two-part polymeric (urethane) foam material, such as the Versi-Foam System by RHH Products, Incorporated (Cudahy, Wis.) or Handi-Foam by Foamo Products, Incorporated (Norton, Ohio), in common use for insulation of structures. These foam component chemicals are supplied in two pressurized tanks which supply the components via separate hoses to a nozzle, wherein the chemicals are mixed to form a foaming liquid. When forming a cast according to the principles of this invention, the foaming liquid material is supplied via fill tubes to the enclosed space of the castable sleeve, wherein the foam expands to fill the sleeve, and solidifies to form the cast. The rapid solidification rate of the mixed liquid/foam material necessitates rapid, uninterrupted flow of the foam to the castable sleeve. The foam is maximally expanded in approximately 30 seconds, and is substantially rigid in less than two minutes. In a preferred embodiment, multiple fill tubes supply the expanding material to multiple locations within the castable sleeve so as to achieve a uniform distribution of foam within the cast. A single tube connected to the nozzle of the foam supply system is connected via a distribution system to the multiple feed tubes. It is necessary that the flow through the individual tubes of the multiple flow tubes be essentially equal to ensure a proper distribution of foam within the castable sleeve and the finished cast. The expanding liquid/foam material in the tubes creates some back pressure on the distribution system so as to ensure that all individual tubes have flow. In some cases, it is helpful to have the individual feed tubes from the manifold system be of a smaller diameter than the tube feeding material to the distribution system so as to create additional back-pressure to ensure even distribution. The tube size must not, however, be so small that it impedes flow of material into the castable sleeve, since it is desirable that most expansion occur in the sleeve. A low flow rate of material to the sleeve can cause the foam to solidify more rapidly in the sleeve, thereby limiting flow of the foam within the sleeve and causing voids in the finished cast.

While multiple fill locations are required to achieve uniform filling of the castable sleeve, it is also desirable to minimize the number of locations. Increasing the number of locations increases the transit time of the material to the sleeve, thereby limiting the flow of foam within the sleeve due to solidification. Also, increasing the number of fill tubes complicates the distribution system and increases the likelihood of unequal flow in the tubes.

The choice of locations for supplying the material is critical, since the foam which is supplied to the sleeve becomes rigid soon after reaching the interior of the sleeve. If the fill locations are in too close proximity to each other and to the axial center of the sleeve, the center portion of the cast will be filled to capacity, but the regions away from the center will be only partially filled since solidification of the foam impedes flow of the foam axially away from the center of the sleeve. Overfilling may occur in the center portion of the sleeve causing the sleeve to fail through rupture or seeping of foam through the sleeve fabric due to localized excessive pressure created by the foam in the sleeve. Conversely, if the fill locations are excessively spaced axially the axially outer portions of the sleeve will be filled but voids created in the center portion of the sleeve. Either non-uniform cast is unacceptable as it does not fully immobilize the patient's extremity, nor does it optimize patient comfort.

In a preferred embodiment, four fill locations are used, the locations being symmetrically placed about the center of the sleeve when viewed in a plan view. Two fill locations on each lateral side are symmetrically displaced axially from the axial center of the sleeve. The number of fill locations can be increased for casts with greater axial length. In other embodiments for immobilizing, for instance, a head or shoulder, non-symmetrical placement of the fill locations in the cast may be required, with the fill locations being optimally selected such that they preferentially fill large void areas between the patient and the cast.

The material from which the castable sleeve is made must allow gaseous products to escape, but must be impermeable to the foam material. The mixed components of the two-part foam are supplied to the interior of the castable sleeve as a mixture of liquid and foam. The initial flow when the valve of the foam supply system is first opened, however, is gaseous. It is essential, therefore, that the castable sleeve material allow these gaseous materials to escape so as to prevent the formation of voids in the cast. In a preferred embodiment the cast material is a stretchable polyester knit fabric having a thread density sufficient to produce impermeability to foam, even when expanded by pressurized foam within the sleeve. In other embodiments other fabrics are used. In yet another embodiment paper is used. Embodiments which use inelastic fabric or paper for the cast material have pleats formed in the distal walls of the sleeve to allow for expansion of the foam material in the sleeve.

In its simplest form, the castable sleeve is a double walled tube having a proximal wall and a distal wall joined at the ends to form a space therebetween which is filled with a quick-set casting material. The patient's body part, such as a limb or extremity, is inserted into the inner portion of the tube, and the tube advanced axially onto the limb until the portion of the limb to be examined is approximately centered axially in the tubular sleeve. The sleeve is then placed in a mold and filled with quickset casting material. This sleeve configuration has two drawbacks. It is frequently difficult and painful to insert an injured limb into the sleeve. Also, a sleeve of this type produces a cast with a monolithic structure which is difficult to remove from the limb after use. The polymeric foam of the preferred embodiment has substantial structural strength requiring that the cast be cut axially for removal from the limb. This cutting procedure is tedious and time consuming, frequently causes patient anxiety, and is also messy since foam material is separated from the cast by the cutting and separation process.

In a more preferred embodiment, the castable sleeve is formed as a double-walled sheet which is wrapped around the subject's body, the longitudinal edges being fastened together so as to form a tube having a proximal wall, a distal wall, and a space therebetween for receiving the casting material. In a preferred embodiment, the fasteners are hook and loop fastener pairs; however, other fastening combinations, such as hooks and eyes, buttons, snaps fasteners, and the like are also contemplated. In one embodiment, a first group of fasteners is placed along the longitudinal edges of the double-walled sheet, and a second group of fasteners is placed in a more or less parallel line, spaced a predetermined distance from the first group of fasteners toward the center of the sleeve when viewed in plan view. In other embodiments, a single group of fasteners may be sufficient. In either instance, when the fasteners are connected, a double walled tubular sleeve having a projecting seam along its longitudinal axis is formed. Furthermore, when the sheet is wrapped about a patient's body to form the tubular sleeve, the laterally opposed fasteners of the second group are joined to form the proximal wall of the tube. The predetermined distance between the laterally opposed fasteners is such that the proximal wall conforms loosely to the patient's body. Joining laterally opposed fasteners of the first group forms the distal wall of the tube, the circumference of the distal wall being greater than that of the proximal wall, but less than the circumference of the inner surface of the mold which surrounds the body and sleeve during casting. When the casting material is injected into the space between the inner and outer walls, the distal wall of the sleeve expands to fill the void between the body and the mold. The circumference of the distal wall of the sleeve must be sufficient to allow complete filling of the mold cavity without expanding the fabric to an extent that the pressurized foam seeps through the fabric. Excessive material, however, is also not desirable, as folds in the material may become trapped in the solidifying foam during the filling process thereby preventing flow of the foam material to all regions of the cast, which, in turn, results in voids and incomplete filling.

In an alternate embodiment, a material having greater elasticity and high thread density is used, such that the proximal and distal walls are approximately equal in circumference. In such instances, only a single group of fastener pairs is needed. The greater elasticity of the sleeve material allows the distal wall to expand maximally without rupturing or allowing foam to seep through the distal wall.

A predetermined shape is imparted to the outer (distal) surface of the cast by a mold having a cavity formed to the complement of the predetermined shape. The predetermined shape is designed for use with a particular coil. For instance, a cast for use with a coil having a cylindrical form will have an outer shape which is more or less cylindrical and of a size which will fit into the coil, substantially filling the volume of the coil. More particularly, the outer shape of the cast has a non-smooth contour, including features formed therein (such as corners and/or projecting edges) which maintain alignment between the subject's body and the axis of the coil and prevent both axial and rotational relative movement. In a preferred embodiment, the cast has polygonal cross-section (for example square, rhomboid, hexagonal, octagonal, etc.), more particularly an irregular polygon having bilateral symmetry. The shape is formed so that two laterally opposed surfaces (e.g., corners or edges) on the lower portion of the cast contact the inner surface of the coil during use, and support the cast within the coil. The shape is also formed so that when the cast is formed in a mold having an upper portion and a lower portion, the upper portion of the mold can be removed after casting, and the cast can be removed from the lower portion of the mold after casting. The mold halves are removably joined during casting to form a complete mold cavity having the complement of the predetermined cast outer shape. Also, in a preferred embodiment, the fill tubes pass from the sleeve within the mold laterally to the outside of the mold in a manner which allows the sleeve with attached fill tubes to be placed in the lower portion of the mold and the upper portion of the mold assembled to the lower portion. In a preferred embodiment, the mold is formed of axially spaced laminations (e.g., thin plates) so that the axial length of the mold can be increased or decreased by adding or removing laminations as required for a particular subject or body part. During filling of the sleeve and solidification of the casting material, gaseous byproducts which pass through the sleeve are able to escape through the spaces between the laminations. In other embodiments, the mold has monolithic walls and a fixed length. In some embodiments, the monolithic walls have a plurality of holes there through to allow the escape of gaseous byproducts. In yet other embodiments, the walls of the mold cavity are formed of a porous material which allows passage of gaseous byproducts.

The castable sleeve has an axial length greater than the axial length of the mold, the extra sleeve length providing a place for the containment of excess foam. Because the size of the body part being cast can vary, the volume of foam required can only be approximated. Typically, an excess foam is injected so as to ensure complete filling of the mold. The excess foam flows axially from the mold into the portion of the sleeve outside the mold. The flow of foam within the mold must, however, be controlled so as to ensure that the mold is completely filled before material begins to flow into the portions of the sleeve outside the mold. In a preferred embodiment, elastic straps or bands may be placed around the sleeve inside the mold at predetermined locations prior to casting. For example, a first pair of bands are may be around the sleeve a predetermined distance axially proximal to the most proximal fill tube and a predetermined distance axially distal to the distal-most fill tube. A second pair of bands may be placed around the sleeve at the proximal and distal ends of the mold. The elastic has a predetermined spring rate such that when the expanding foam completely fills the volume between a pair of elastic bands, the bands stretch so as to allow the foam to fill the adjacent portion of the sleeve. When the portion of the sleeve within the mold is completely filled, such that the void between the body and the mold is filled, the bands at the proximal and distal ends of the mold allow excess foam to fill the portions of the sleeve lying outside the mold. Hence, the elastic bands create communicating chambers that sequentially fill. In this manner, complete filling of the sleeve portion within the mold may be achieved, and excess foam may be contained without rupturing of the sleeve. In other embodiments, the elastic bands may be integrated with the sleeve assembly. In another embodiment, the material of the sleeve may have a non-uniform elasticity, such that the resistance to deformation is greater in the proximal and distal portions of the sleeve than at the central portion in the region of the fill tubes.

The filled sleeve has a seam along its upper surface formed by the meeting of the lateral ends of the sleeve sheet at the fasteners. This seam is somewhat irregular due to localized inequalities in the fill rate on opposite sides of the seam.

Because of high localized pressures within the sleeve, and unequal expansion of the sleeve material, some seeping of foam material through the sleeve frequently occurs. This material causes adhesion between the sleeve and any adjacent material. To prevent adhesion between the sleeve and the mold, a foam-impermeable barrier may be placed between the mold and the sleeve. In a preferred embodiment, the impermeable barrier is a close-weave fabric. For example, in use, a first barrier sheet of the fabric may be placed in the lower portion of the mold before placement of the sleeve in the mold. A second sheet is then placed around the upper portion of the sleeve in the mold prior to assembly of the upper portion of the mold to the lower portion. When the casting process is complete, the impermeable sheets may be removed from the cast. Adhesion between the impermeable sheets and the cast is such that the sheets are easily removed. In some embodiments the fabric is elastic; in others, it is inelastic.

Seepage of foam through the proximal wall of the sleeve is also possible. In a preferred embodiment, a foam-impermeable barrier is provided on the subject's body, for example, using a thin plastic film which is wrapped around the body prior to casting. In other embodiments, a plastic sleeve may be placed on the body. In yet other embodiments, a foam-impermeable fabric may be used.

The reaction of the component chemicals to produce the foam is exothermic. Heat produced by the reaction tends to heat the patient's body. Because the thermal mass of the foam is low, the heating of the body is insufficient to harm the subject and generally does not cause discomfort. However, it may be necessary for some subjects to have a thermal barrier which decreases heating of the body. Accordingly, in some embodiments, a sleeve formed of a knit fabric or other thermally insulating material is placed on the body prior to casting. In some embodiments, the sleeve is placed under the foam-impermeable barrier on the patient. In other embodiments, the sleeve is placed over the foam-impermeable barrier on the subject's body. In yet other embodiments, a thermal barrier sleeve is not used.

In use, the subject's body is covered with a plastic film material and a thermal barrier sleeve is applied, if required. A first foam-impermeable sheet is placed in the lower portion of the mold. The castable sleeve sheet assembly with the attached fill tubes is placed in the lower portion of the mold with the fasteners upward and the fill tubes extending outward from the lateral upper lateral edges of the lower mold portion. The plastic-wrapped body (e.g., a limb) is placed in the lower portion of the mold on top of the castable sleeve sheet assembly. The sheet assembly is fastened along the top of the body, the laterally opposed fasteners of the second group of fasteners being joined to form the proximal wall of the sleeve, and laterally opposed fasteners of the first group of fasteners being joined to form the distal wall of the sleeve. An elastic band is placed around the sleeve a short distance proximal from the proximal most pair of fill tubes. A second elastic band is placed around the sleeve a short distance distal to the distal-most pair of fill tubes. A third elastic band is placed around the sleeve approximately even axially with the proximal end of the mold. A fourth elastic band is placed around the sleeve approximately even axially with the distal end of the mold. The fill tubes are positioned in lateral passages joining the inner and outer surfaces of the mold. A second impermeable sheet is placed in the mold on top of the sleeve assembly. The mold upper portion is positioned on top of the lower portion and removably secured. In a preferred embodiment, portions of the upper and lower assemblies mesh at their mating surfaces in much the same manner as a hinge, and rods are inserted into axial holes in the interlocking portions.

The nozzle of a two-part foam system is attached to a main fill tube, which is attached via a distribution system to the individual fill tubes of the sleeve assembly. Supply valves of the foam system are opened so that the two foam chemical components are supplied to the nozzle for mixing. The mixed chemicals, now rapidly expanding to form foam, are supplied to the main fill tube, and therefrom to the individual fill tubes of the sleeve assembly. The rapidly expanding foam fills the inner volume of the sleeve which expands so as to fill the void between the patient's body and the mold. The first elastic band and second elastic band define a first region which is filled by the expanding foam. When this first region is filled, pressure from the expanding foam causes the first elastic band to stretch, thereby allowing expanding foam to flow into a second proximal region of the sleeve between the first elastic band and the third elastic band. More or less simultaneously, pressure from the rapidly expanding foam causes the second elastic band to expand thereby allowing expanding foam to flow into a third region of the sleeve between the second elastic band and the fourth elastic band. In this manner the axially-mid portion of the cast is filled completely prior to filling of the more proximal and distal portions of the cast so as to ensure complete filling of the void in the region between the subject's body and the mold. To ensure complete filling of the void, additional expanding foam is injected into the mold space. Pressure from this rapidly expanding foam supplied to the first portion of the sleeve and therefrom to the second and third portions causes the third elastic band to stretch thereby allowing expanding foam to fill a fourth portion of the sleeve proximal to the second portion and proximal to the proximal end of the mold. In a similar manner, and more or less simultaneously, expanding foam in first and third portions of the sleeve causes the fourth elastic band to expand allowing foam to fill a fifth region distal to the third region and distal to the distal end of the mold. This controlled flow of foam to the fourth and fifth portions of the sleeve ensures that the void between the subject's body and the mold wall is completely filled, and that overfill does not cause rupture of the sleeve and escape of the expanding foam material.

When the foam material is fully solidified, generally in three minutes or less, the rods joining the mold upper and lower portions are removed and the top portion of the mold is removed. The second impermeable sheet is removed from the top of the cast. The subject's limb with the attached cast is removed from the bottom half of the mold. The fill tubes are removed from the cast. In a preferred embodiment the fill tubes are affixed to the sleeve by a means which allows the tubes to be easily detached from the cast by pulling. The portions of the fill system are discarded.

The subject's body is then positioned in the lower half of a coil of an MRI machine, the locating portions of the lower portions of the cast resting on the inner surface of the coil. An expandable or resilient member is placed on top of the cast, and the upper half of the coil removably affixed to the lower half of the coil. In a preferred embodiment, an expandable member in the form of an inflatable vessel is used. The vessel is inflated via a valve such that inflation of the vessel is maintained after the vessel is inflated. Downward and expansion force exerted by the vessel on the cast immobilizes the cast within the coil, the alignment of the cast being determined by the locating surfaces of the cast.

After completion of the exam, the upper portion of the coil is removed. The expandable/resilient member is removed and stored for future use. The subject's limb (with cast) is removed from the coil. The cast is removed from the subject's body by applying a lateral separating force to opposite sides of the upper seam of the cast causing the seam to separate and the lower portion of the cast to fracture. The sleeve does not rupture during removal and contains the foam material thus avoiding mess, exposure and cleanup. The lateral separating force can be applied manually by grasping excess material on opposite sides of the seam and pulling. Alternatively, a spreading tool can be inserted into the seam.

Other embodiments are anticipated. For instance, the castable sleeve can be made from other materials which allow the passage of gaseous products but which are impermeable to foam. Paper products with these characteristics may be used, the distal wall of the sleeve being formed with pleats or folds to allow expansion during the casting process. Inelastic fabrics may also be used, the distal wall of the sleeve being formed with pleats to allow expansion during casting. Polymeric materials with controlled porosity may also be used, the distal walls being formed to suit the elastic properties of the materials, inelastic materials requiring pleats or folds for expansion.

The expandable member placed between the cast upper surface and the inner surface of the coil may be replaced by a resilient member which is compressed when the upper portion of the coil is mounted to the lower portion. In other embodiments, wherein the expandable or resilient member is eliminated, the upper surface of the cast having deformable (compressible) portions which compress when the upper portion of the coil is mounted to the lower portion. These portions inelastically deform, but also have sufficient elasticity to ensure that the cast is held motionless within the coil. In other embodiments, the deformable portions are distributed about the cast outer surface so as to locate and immobilize the cast within the coil

The terms “limb” and “extremity” used in the description of the invention herein disclosed are not meant to restrict in any way the scope of the invention. The invention herein disclosed is applicable to the immobilization of arms, legs, feet, neck, abdomen, hands, etc. of any suitable subject. It is also applicable to the immobilization of heads, the cast being formed to a shape which envelopes the upper portion of the head and forms locating surfaces, the location of fill tubes and elastic bands being such that complete filling of the cast is assured. The invention may also be used to immobilize shoulders, the cast surrounding the shoulder and arm so as to provide complete immobilization in a shoulder coil.

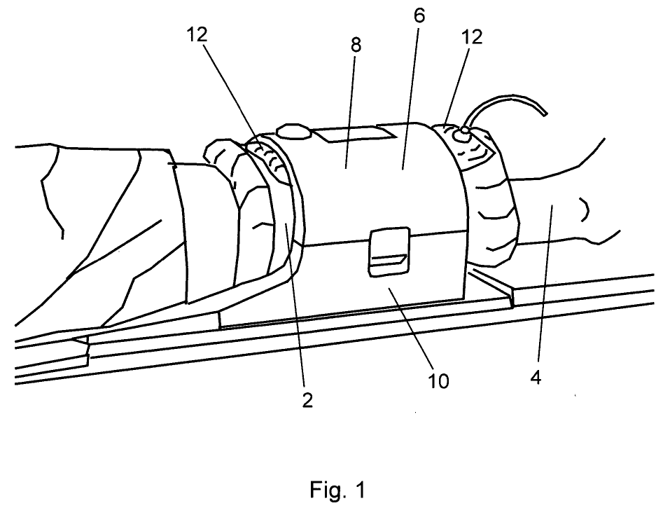

Referring to FIG. 1, which depicts a method and system constructed in accordance with the invention herein, disclosed for immobilizing a subject's limb in an MRI for examination, cast 2, formed of a quick-setting casting material, surrounds subject limb 4. Cast 2 is rigidly, removably positioned within coil 6, comprised of a removable upper coil portion 8 and fixed lower portion 10. Inflatable member 12, removably positioned in the gap between cast 2 and upper coil portion 8, applies a downward force to cast 2 so as to prevent movement of the cast within the coil.

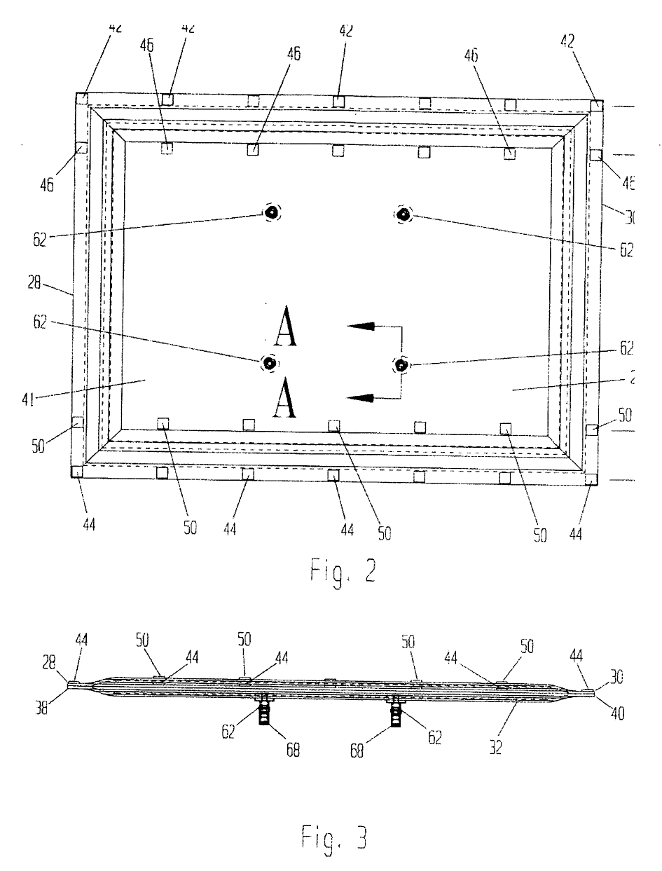

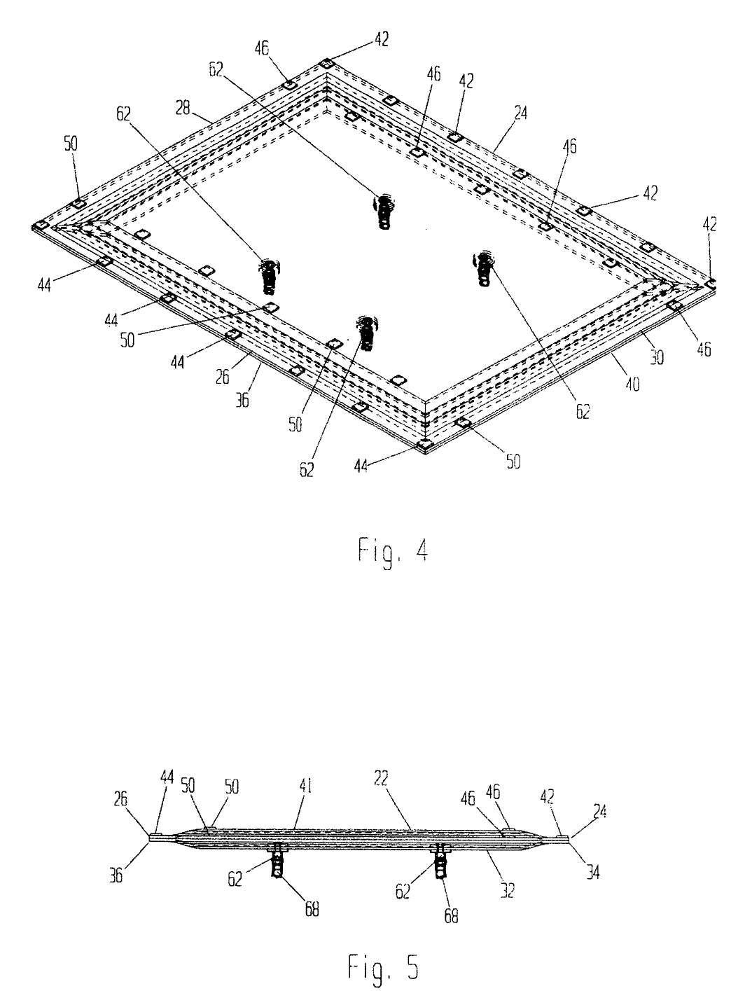

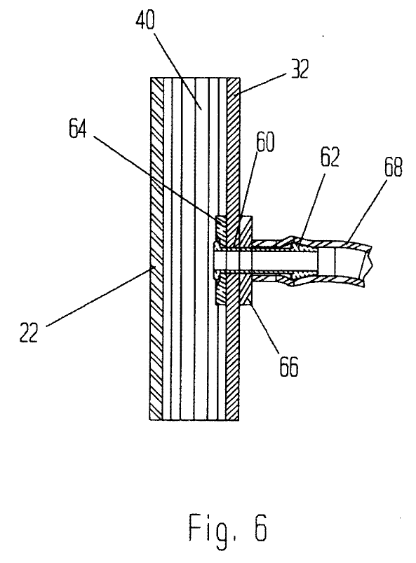

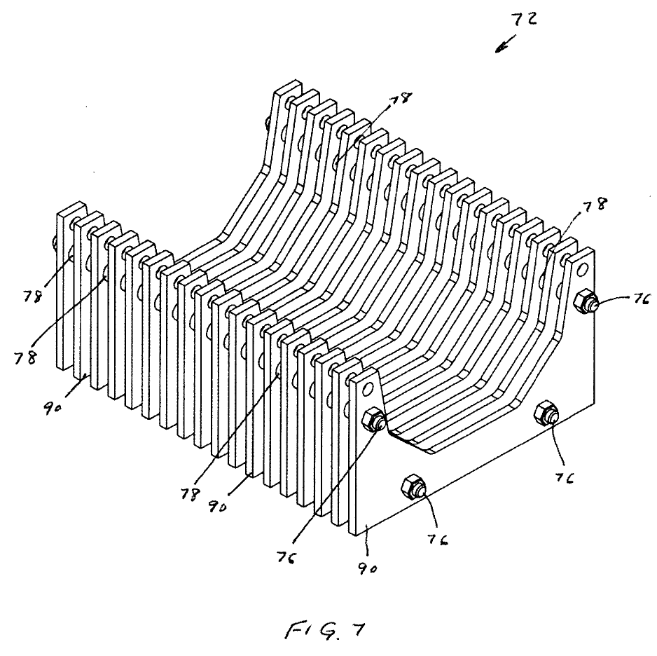

FIGS. 2 through 6 depict a castable sleeve assembly 20 having an upper proximal sheet 22 with a first lateral edge 24, a second lateral edge 26, a distal edge 28 and a proximal edge 30, and a lower, distal sheet 32 with a first lateral edge 34, a second lateral edge 36, a distal edge 38 and a proximal edge 40. Upper sheet 22 and lower sheet 32 are preferably made of foam-impermeable fabric. In a preferred embodiment, the fabric is an elastic polyester knit material having a thread density sufficient to provide impermeability to expanding polymeric foam material. Edges 22 and 32 are joined by stitching or a suitable adhesive material. Edge pairs 24 and 34, 26 and 36, and 28 and 38 are joined in the same manner so as to form an enclosed space 40. On upper surface 41 of proximal sheet 22, first lateral fasteners 42 are adjacent to first lateral edge 24. Second lateral fasteners 44 are adjacent to second lateral edge 26, fasteners 42 and 44 forming fastener pairs such that laterally opposite pairs of fasteners 42 and 44 can be removably joined. In a preferred embodiment, fasteners 42 and 44 are hook and loop fasteners; however, as noted above, other fasteners combinations may be used. Third lateral fasteners 46 are displaced a distance 48 from first lateral fasteners 42. Fourth lateral fasteners 50 are displaced a distance 52 from second lateral fasteners 44, distances 48 and 52 being approximately equal. Fasteners 46 and 50 form fastener pairs such that laterally opposite pairs of fasteners 46 and 50 can be removably joined. In a preferred embodiment, fasteners 46 and 50 are hook and loop fasteners, though, again, alternate fastening means are contemplated. Lower, distal sheet 32 has formed therein openings 60 in which tube connectors 62 are mounted. Inner compressible washers 64 and outer compressible washers 66 provide a seal between sheet 32 and connectors 62. Fill tubes 68 mounted to connectors 62 retain sheet 32 between washers 64 and 66. Other methods for connection of the fill tubes and fittings to the distal sheet are anticipated. For instance, connectors 62 may be attached to sheet 32 by an adhesive, or may have provisions for connection to sheet 32 by stitching or a mechanical fastening means. In use, fasteners 42 are joined to fasteners 44 to form a tubular member with a proximal wall formed of upper sheet 22 which conforms loosely to a patient's limb. Fasteners 46 are joined to fasteners 50 so as to form the distal wall of the tubular member.

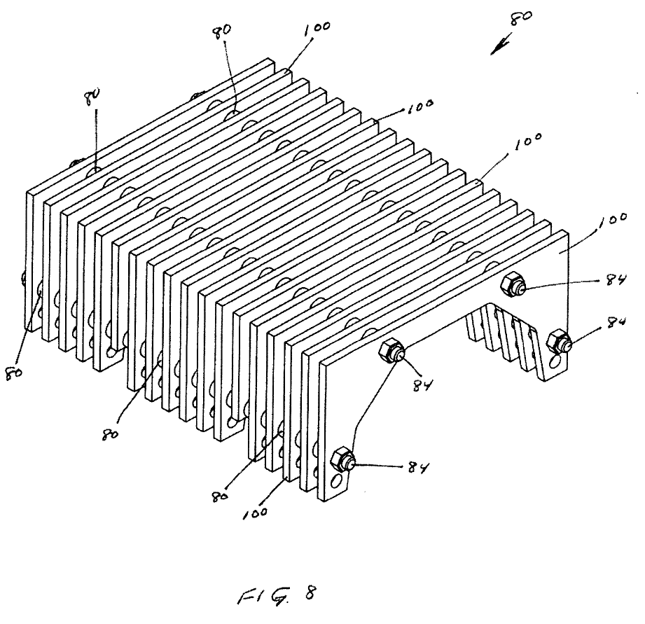

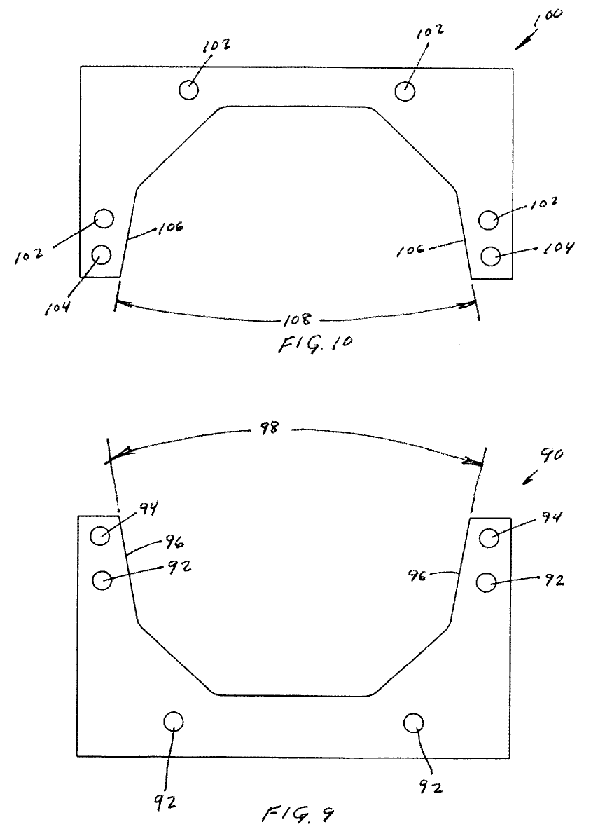

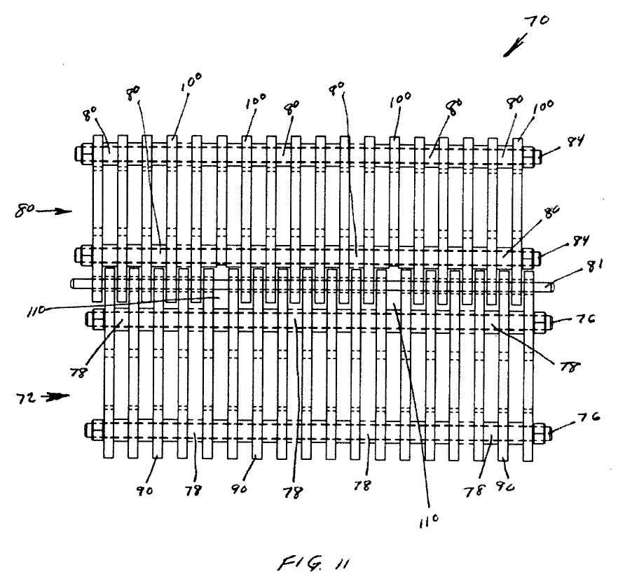

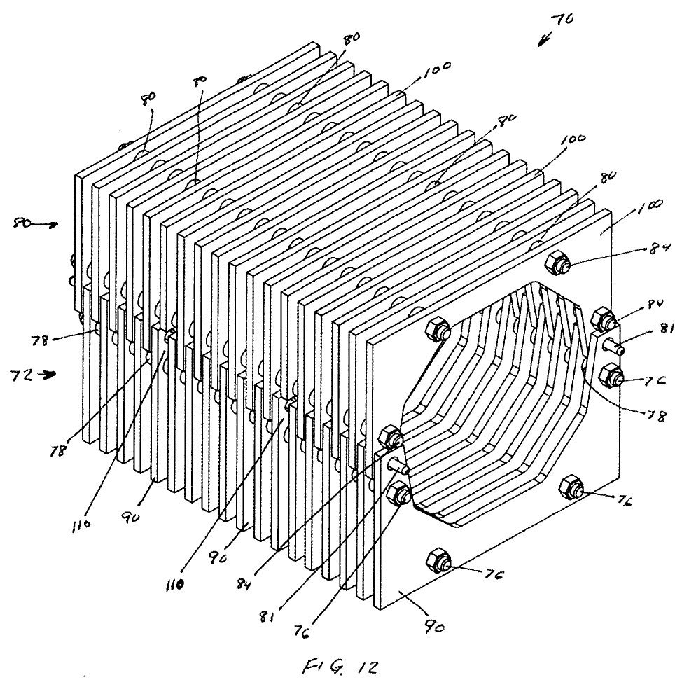

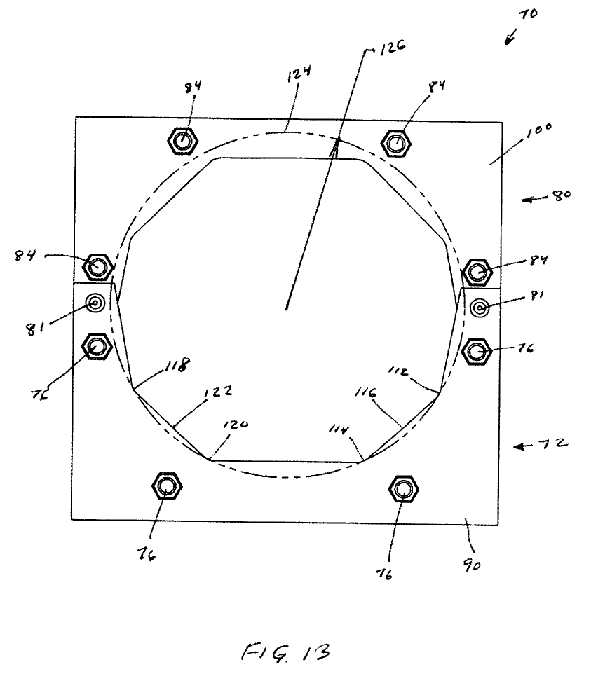

Referring now to FIGS. 7 through 13, mold 70 has a lower subassembly 72 formed of a plurality of laminations 90 joined by connectors 76 and separated by spacers 78. Mold upper subassembly 80 is formed of laminations 100 joined by connectors 84 and separated by spacers 80. As best seen in FIG. 9, lower lamination 90 has holes 92 for connecting to spacers 78 and other laminations 90 so as to form lower subassembly 72. Lower lamination 90 also has holes 94 for removable mounting of lower subassembly 72 to upper subassembly 80. Lower subassembly lamination 90 is bilaterally symmetrical. Upper lateral inner surfaces 96 form an acute angle 98. Referring now to FIG. 10, upper subassembly lamination 100 has holes 102 for connecting to spacers 78 and other laminations 100 to form upper subassembly 80. Upper lamination 100 also has holes 104 for removable mounting to upper subassembly 80 to lower subassembly 72. Lower lateral inner surfaces 106 form an acute angle 108. Upper subassembly lamination 100 is bilaterally symmetrical. Referring again to FIGS. 7 through 13, mold 70 has openings 110 formed in the side walls to allow fill tubes 68 (FIGS. 2 through 6) to pass there through. Upper subassembly 80 and lower subassembly 72 are removably joined by connector rods 81. As best seen in FIG. 13, mold 70 has an inner cavity cross-section forming a polygon. Upper radius 112 and lower radius 114 of surface segment 116, and upper radius 118 and lower radius 120 of surface segment 122 are tangent to circle 124 of radius 126.

In the context of the present invention, the mold cross-section may be a regular or irregular polygon. However, to maximize the space for the subject's limb within the cast, it is often preferred to utilize a mold having an irregular cross-section. In other embodiments, the cross-section may comprise a series of connected linear and/or curvilinear segments, not necessarily forming continuous smooth tangencies.

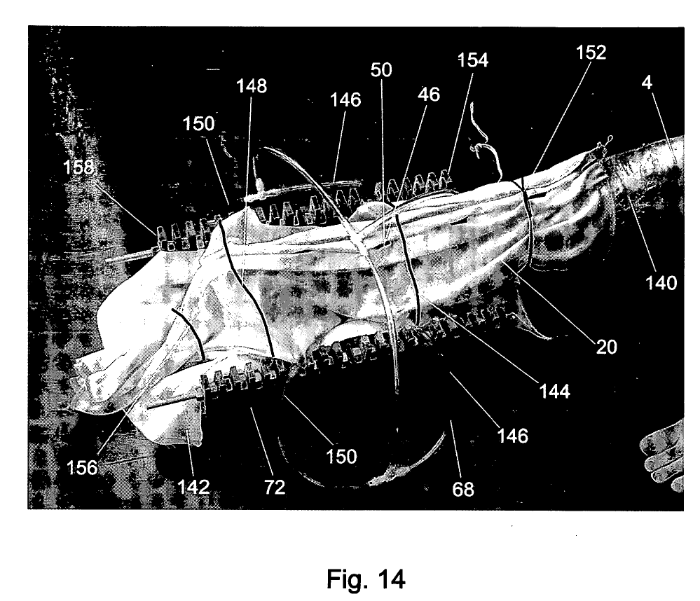

Referring now to FIG. 14, depicting a limb in the process of preparation for casting, plastic wrap 140 is applied to patient limb 4. First impermeable sheet 142 is placed inside mold lower subassembly 72. Castable sleeve assembly 20 is positioned in lower subassembly 72, proximal surface 22 facing upward and fill tubes 68 extending to the outside of the subassembly. Limb 4 is placed on top of sleeve assembly 20, and fasteners 42 and 44 are joined to form a proximal wall, and fasteners 46 and 50 joined to form a distal wall. First elastic band 144 is placed around the sleeve proximal to proximal fill tubes 146. Second elastic band 148 is placed around the sleeve distal to distal fill tubes 150. Third elastic band 152 is placed around the sleeve in the region of proximal end 154 of lower subassembly 72. Fourth elastic band 156 is placed around the sleeve in the region of distal end 158 of lower subassembly 72.

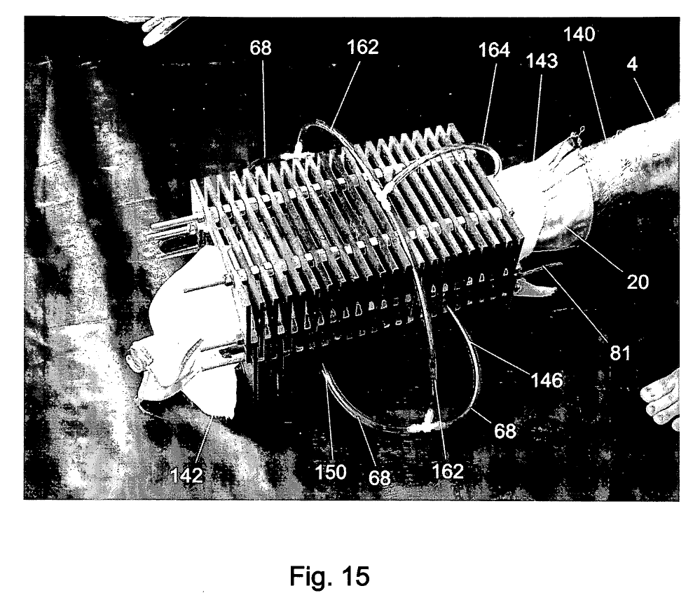

Referring to FIG. 15, second impermeable sheet 143 is placed on top of sleeve assembly 20 and mold upper subassembly 80 is mounted to lower subassembly 72 using connector rods 81. Fill tubes 68 are connected via secondary fill tubes 162 to primary fill tube 164. Primary fill tube 164 is connected to the mixing nozzle of a foam chemical component supply system equipped with a valve, 166. Opening valve 166 for a predetermined length of time injects the proper amount of mixed components to space 40 (FIGS. 2 through 6) of sleeve assembly 20.

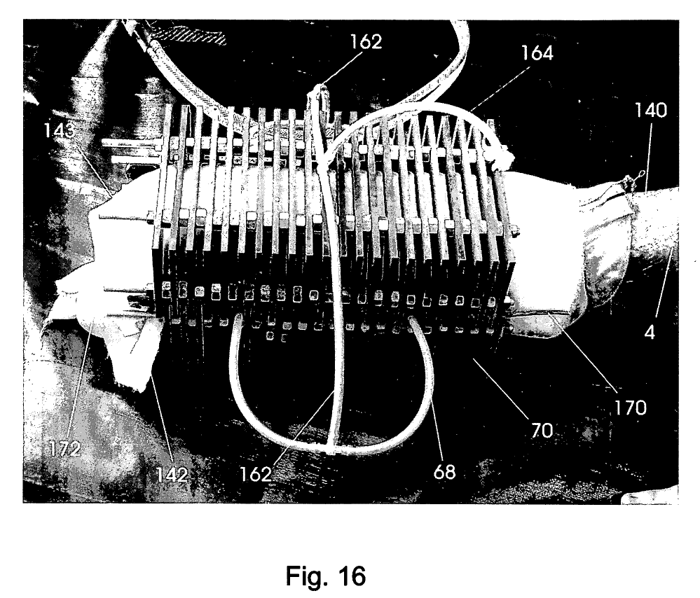

As best seen in FIG. 16, which depicts a mold with a castable sleeve therein, the castable sleeve being filled with foam which has solidified to form a cast, when mixed foam components are injected into space 40 (FIGS. 2 through 6), foam fills the void between sleeve assembly 20 and mold 70 completely in the manner previously herein described. That is, the foam first fills the region of the void between first elastic band 144 and second elastic band 148 (FIG. 14), then the regions between elastic bands 144 and 152, and between bands 148 and 156. Excess foam material then flows into a first portion 170 of the sleeve proximal to band 152, and portion 172 distal to band 156.

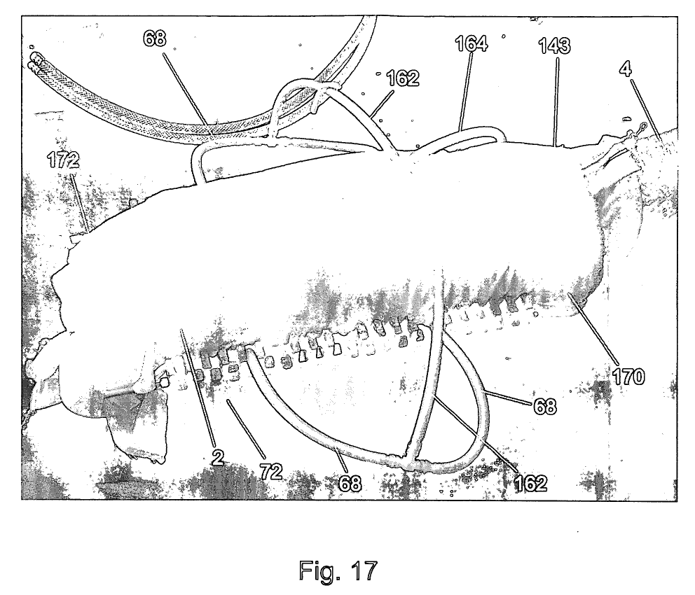

FIG. 17 shows a finished cast 2 prior to removal from mold lower subassembly 72. Fill tubes 68, secondary fill tubes 162 and primary fill tube 164 are attached to cast 2. Upper impermeable sheet 143 is adhering to the upper surface of the cast covering elastic bands 144, 148, 152 and 154 shown in FIG. 14. Excess foam material has solidified in portion 170 of the sleeve proximal to band 152, and portion 172 distal to band 156.

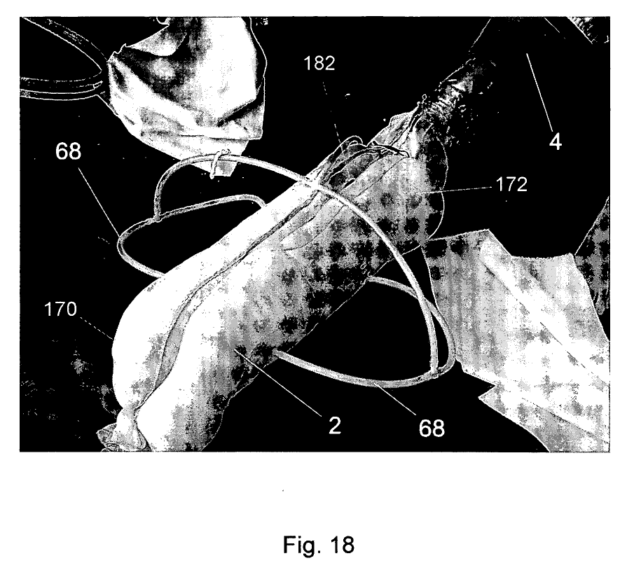

FIG. 18 shows finished cast 2 on a patient's foot. Fill tubes 68 are subsequently removed from the cast prior to placement in an MRI coil as shown in FIG. 1. Excess foam material in portions 170 and 172 is evident. Seam 182 in the upper portion of cast 2 is formed by lateral edges of sleeve assembly 20 (FIG. 14) where joined by fasteners 42, 44, 46 and 50 (FIGS. 2 through 4). After use cast 2 is removed form the patient by applying a lateral separating force to seam 182 so as to open the seam and cause fracturing of cast 2 along its bottom portion.

Referring again to FIG. 1, cast 2 and limb 4 are placed in coil 6 by removing coil upper portion 8 and placing the limb and cast in the coil lower portion 10. Inflatable vessel 12 is placed on top of cast 2 and then upper portion 8 is mounted to lower portion 10. Vessel 12 is then inflated to exert a downward force on cast 2.

As seen in FIG. 19, showing a section view of the coil, cast and limb portion of FIG. 1, limb 4 is encased within cast 2 which is located and immobilized within coil 6. Vessel 12, on top of cast 2 and below coil upper portion 8 forces locating features on the lower portion of cast 2 into contact with coil lower portion 10. Coil 10 has a cylindrical inner surface 190 of radius 192 equal to radius 126 (FIG. 13). Radii 194 and 196 of cast surface 198 formed by radii 118 and 120 of surface 122 of mold lower portion 72 (FIG. 9), and radii 200 and 202 of cast surface 204 formed by radii 112 and 114 of surface 116 of mold lower portion 72 (FIG. 9) are in contact with surface 190 of coil lower portion 10. Contact between radii 194, 196, 200, and 202 and surface 190 aligns the axis of cast 2 with coil 6. Frictional forces between the radii and surface 190 and between vessel 12 and surface 190 prevent rotation and axial movement of cast 2.

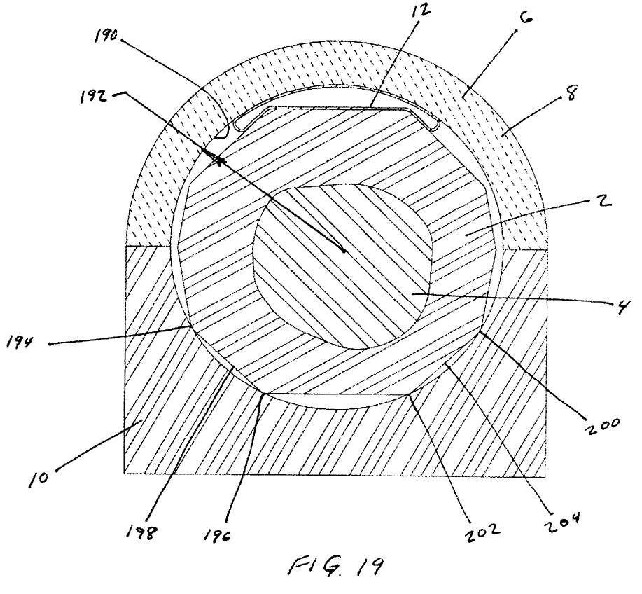

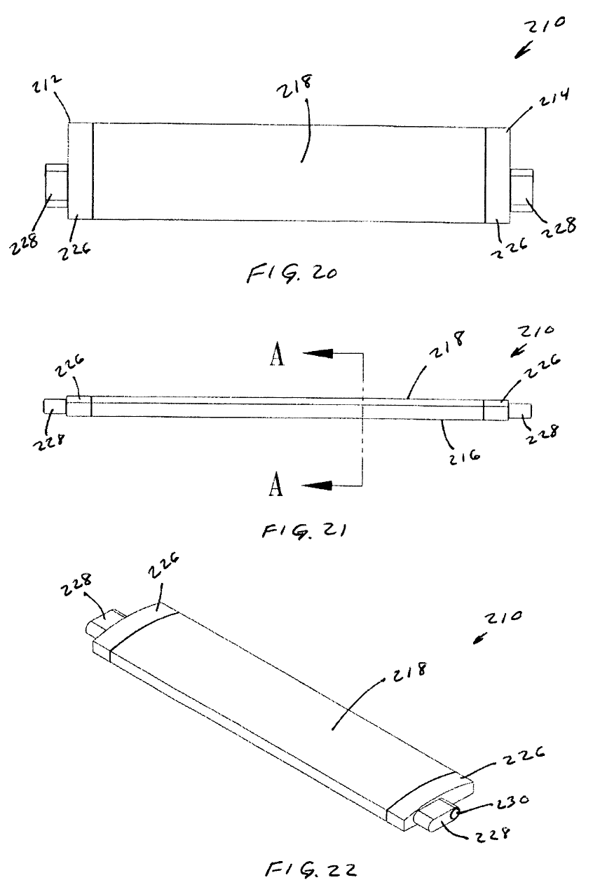

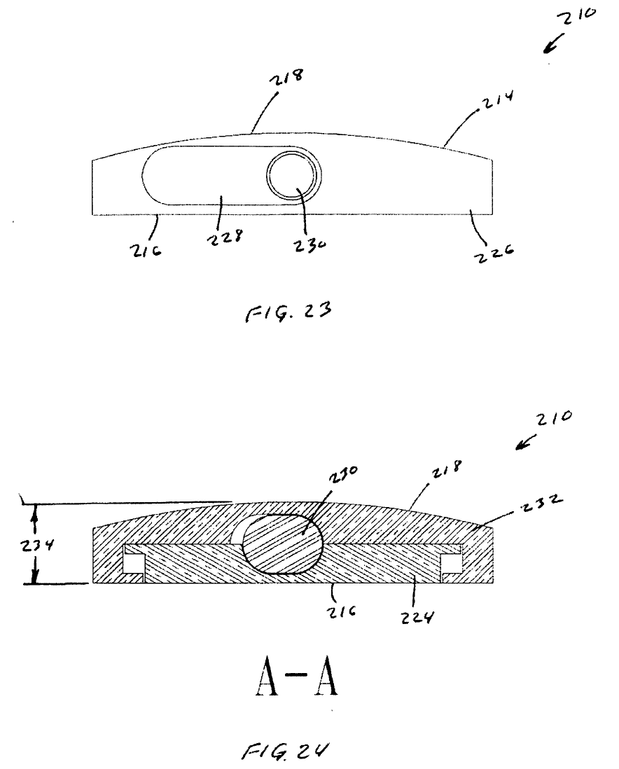

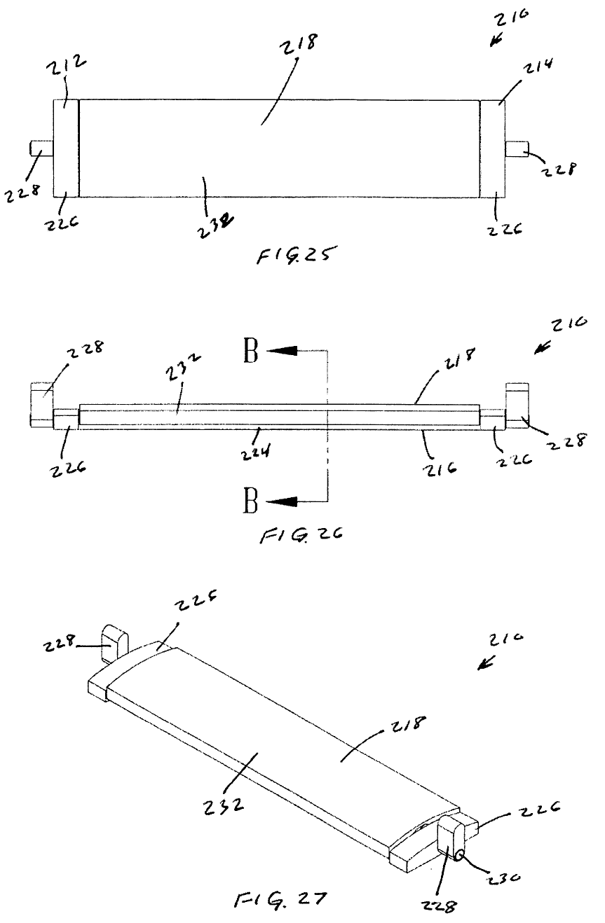

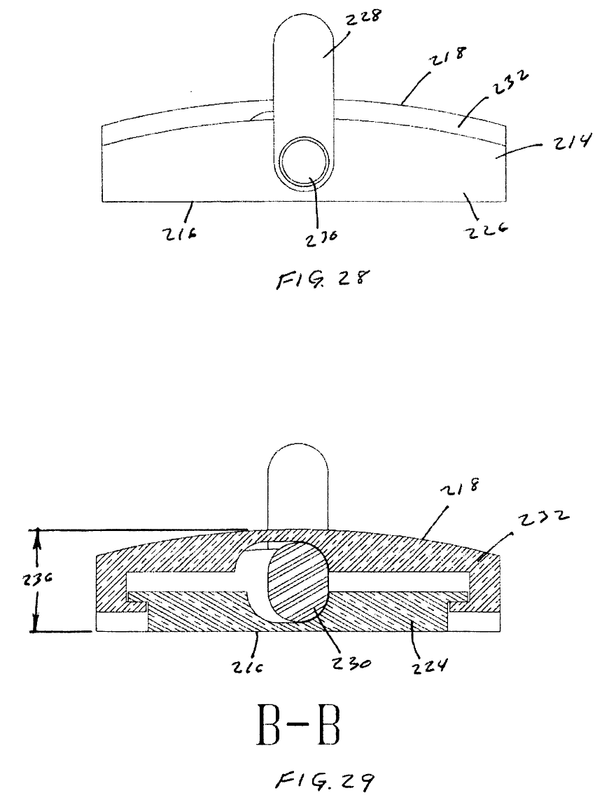

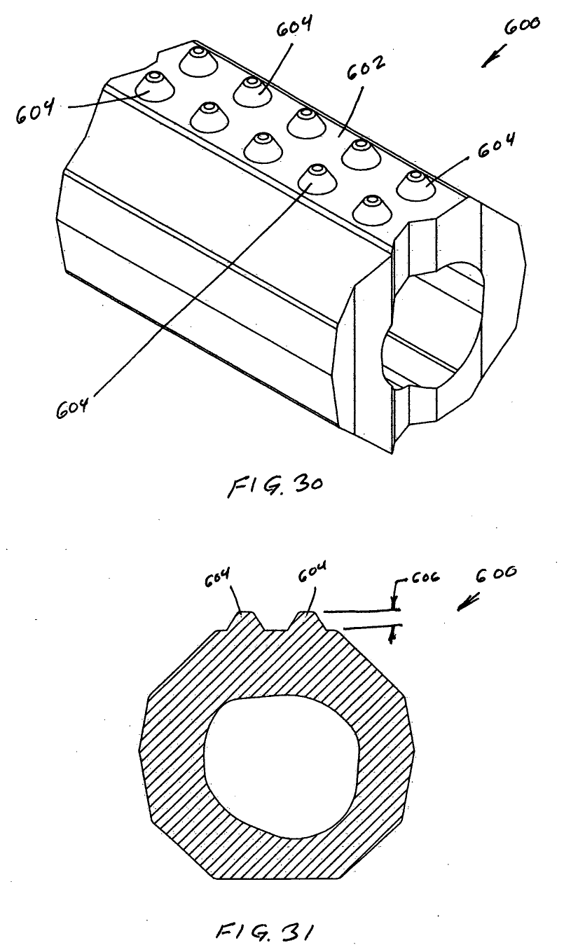

In another embodiment, inflatable vessel 12 is replaced with an expandable mechanical device. As seen in FIGS. 20 through 24, depicting the expandable device 210 in its retracted position, device 210 has a first end 212, a second end 214, a bottom surface 216 and a cylindrical top surface 218 of radius 220. Device 210 has a lower subassembly 222 formed of base 224, end caps 226, handles 228 and cam 230. As best seen in FIG. 24, upper member 232 is movably affixed to lower subassembly 222 such that member 232 can be moved to a second, extended position by rotation of cam 230. Handles 228 are affixed to cam 230 such that cam 230 can be rotated thereby. As seen in FIG. 24, device 210 has a height 234 in its retracted position. Referring now to FIGS. 25 through 29 showing device 210 in its extended position, handles 228 are rotated to the position shown thereby rotating cam 230 so as to displace upper member 232 upward to a height 236 (See FIG. 29).

In use, cast 2 and limb 4 are placed in coil 6 by removing coil upper portion 8 and placing the limb and cast in the coil lower portion 10 (See FIG. 1). Expandable device 210 in its retracted position is placed on top of cast 2 and then upper portion 8 is mounted to lower portion 10. Handles 228 are rotated to their upward position thereby rotating cam 230 and increasing the height of device 210 to height 236 so as to exert a downward pressure on cast 2 thereby preventing movement of cast 2 within coil 6. In another embodiment, top surface 218 of device 210 is coated with a slip resistant material such as neoprene so as to increase the resistance to relative motion of cast 2 relative to coil 6.

In another embodiment downward force is exerted on the cast 2 by a resilient member, such as a compressible spring or deformable elastomeric layer, placed on top of cast 2 prior to affixing removable upper portion 8 of coil 6 to fixed lower portion 10.

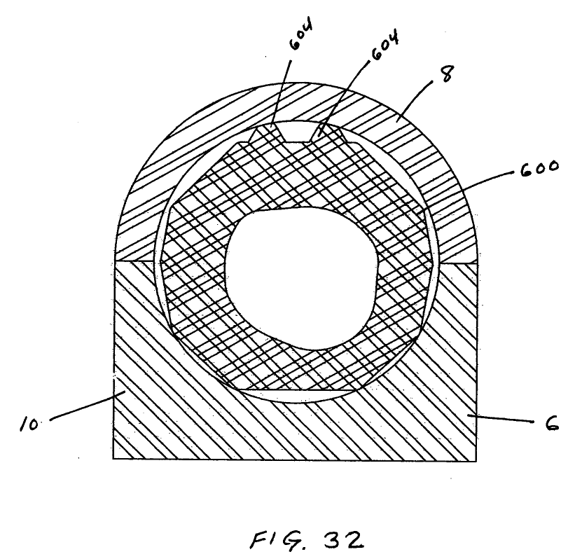

In another embodiment, the cast has compressible protuberances formed on the upper portion of the cast. The protuberances are of a height which causes them to be inelastically deformed when the upper portion of the coil assembly is mounted to the lower portion with the cast in place in the coil. Referring to FIGS. 30 and 31, cast portion 600 has formed in its upper surface 602 a plurality of protuberances 604 of height 606. Referring to FIG. 32, which depicts a section view of cast portion 600 mounted in coil 6, when upper portion 8 of coil 6 is mounted to lower portion 10, protuberances 604 are deformed by inner surface 190 thereby producing a downward force on cast portion 600. This downward force prevents angular or axial movement of cast 600 relative to coil 6. In other embodiments the protuberances are ribs, ridges or other locally raised portions of upper surface 602. In still other embodiments the protuberances are distributed over other external surfaces of the cast so that deformation of the protuberances provides location of the cast within the coil, and compressive force against the wall of the coil so as to prevent movement of the cast within the coil.

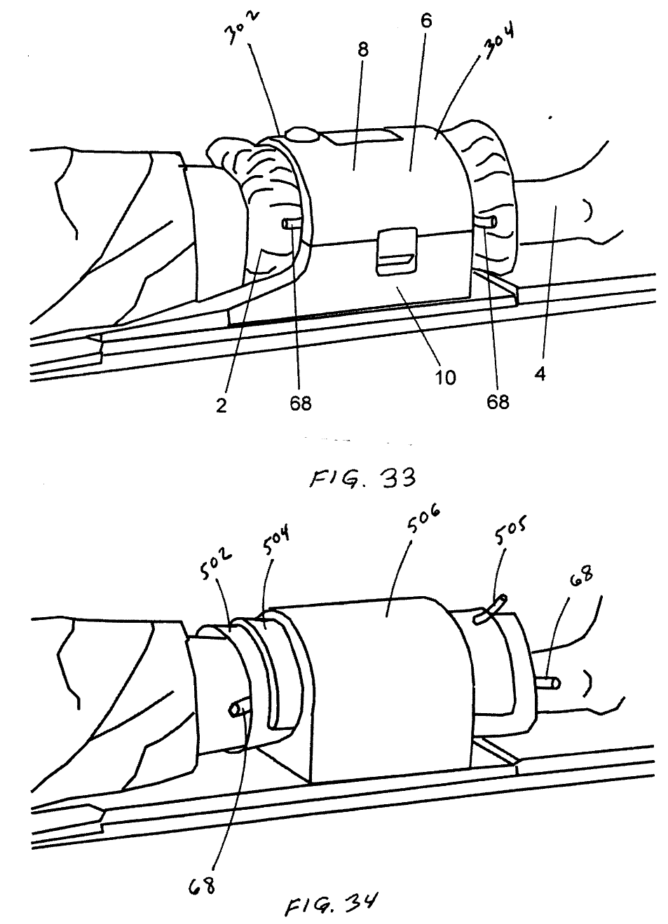

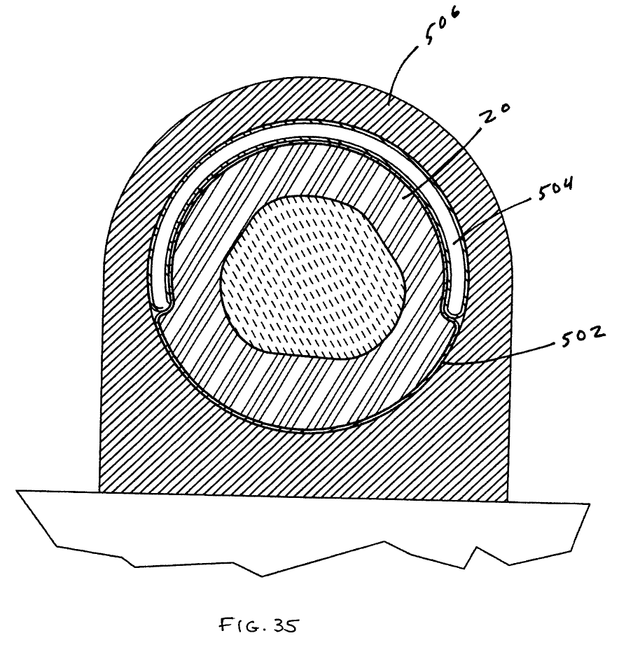

In yet another embodiment, shown in FIG. 33, the cast is formed in place, in the interior space of the coil rather than in a mold, so as to minimize the need to move injured limbs. Because the cast is formed in place, it conforms to the interior of the coil. Expansion of the foam produces a compressive contact force between the cast outer wall and the inner wall of the coil thereby preventing movement of the cast in the coil. A casting sleeve assembly 20 like that of previous embodiments is used, however the fill tubes 68 exit the distal and proximal ends of the coil 6, after which they are joined to fill tubes 162 (FIGS. 15 through 18). Sleeve assembly 20 is formed in the same manner as the previous embodiments (see FIGS. 2 through 6) and is applied to the patient and used in the same manner. The limb is prepared and the casting sleeve applied in the same manner as shown in FIGS. 15 through 18 for the previous embodiment. Plastic wrap 140 is applied to patient limb 4. First impermeable sheet 142 is placed inside lower portion 10 of coil 6. Castable sleeve assembly 20 is positioned in lower portion 10, proximal surface 22 facing upward and fill tubes 68 extending axially to the outside of lower portion 10. Limb 4 is placed on top of sleeve assembly 20, and fasteners 42 and 44 are joined to form a proximal wall, and fasteners 46 and 50 joined to form a distal wall. First elastic band 144 is placed around the sleeve proximal to proximal fill tubes 146. Second elastic band 148 is placed around the sleeve distal to distal fill tubes 150. Third elastic band 152 is placed around the sleeve in the region of proximal end 302 of lower portion 10 of coil 6. Fourth elastic band 156 is placed around the sleeve in the region of distal end 304 of lower portion 10. Second impermeable sheet 143 is placed on top of sleeve assembly 20 and coil upper portion 8 is mounted to lower portion 10 of coil 6. Fill tubes 68 are connected via secondary fill tubes 162 to primary fill tube 164. Primary fill tube 164 is connected to the mixing nozzle of a foam chemical component supply system equipped with a valve, 166. Opening valve 166 for a predetermined length of time injects the proper amount of mixed components to space 40 (FIGS. 2 through 6) of sleeve assembly 20. Filling occurs in the same sequential manner as the previous embodiment, the flow of the expanding liquid/foam material being determined by elastic bands 144, 148, 152 and 156. Because the foam expands to fill the entire the inner space of coil 6, an expandable vessel or other expandable means are not required. Expansion of excess foam within sleeve assembly 20 outside proximal end 302 and distal end 304 of coil 6 provides additional resistance to movement of the cast and limb. In another embodiment first impermeable sheet 142 and second impermeable sheet 143 are replaced by a single, inelastic, impermeable sheet having fasteners along its lateral edges so that the sheet lateral edges can be joined to form a tubular sleeve having a diameter slightly larger than the diameter of the interior of coil 6. The length of this impermeable sleeve is longer than the axial length of coil 6, but shorter than the axial length of castable sleeve assembly 20. During use, excess foam which fills the portions of sleeve assembly 20 proximal to proximal end 302 of coil 6 and distal to distal end 304 of coil 6 is confined within the tubular sleeve formed by the inelastic impermeable sleeve. This confinement of the excess foams extends the length of the cast beyond the length of coil 10 providing additional immobilization of the limb confined therein. Removal of the cast from the patient is performed in the same manner as in previous embodiments.

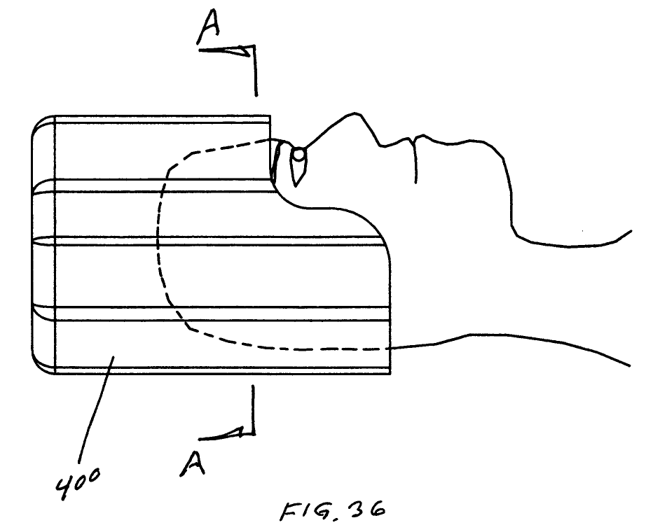

The cast-in-place embodiment depicted in FIG. 33 is preferably used with imaging coils that have a removable portion which aids in placement of a limb or body portion in the coil prior to imaging and removal from the coil after imaging. Some coils do not have removable portions, particularly those coils used with imaging systems designed primarily for the examination of limbs. An embodiment of the invention herein disclosed, intended for cast-in-place use particularly with one-piece coils (those which do not have a removable portion) is depicted in FIGS. 34 and 35. While the previously described cast-in-place embodiments will locate a body portion reliably within a one-piece coil, removal of the cast from the coil can be problematic. Axial removal of a cast-in-place cast from a one-piece coil is difficult since the cast conforms to the inner contours of the coil and is in contact with the interior walls, and because the excess foam which flows from the proximal and distal ends of the coil tends to form masses which are larger in size than the diameter of the coil. In the embodiment of FIGS. 34 and 35, an expandable (collapsible) member is placed in the coil prior to forming of the cast such that a first portion of the cast conforms to the inner contours of the coil, and a second portion is in contact with and conforms to the expandable member. The cast is removed from the coil by collapsing the expandable member so as to create clearance between the cast and the coil interior. Also, a tubular member formed from an inelastic sheet material prevents the excess foam from forming large masses exterior to the coil. In a preferred embodiment shown in FIGS. 30 and 31, the expandable member is an inflatable vessel. Referring to the figures, a casting sleeve assembly 20 like that of previous embodiments is used, however the fill tubes 68 exit the distal and proximal ends of the coil 6, after which they are joined to fill tubes 162 (FIGS. 15 through 18). Sleeve assembly 20 is formed in the same manner as the previous embodiments (see FIGS. 2 through 6) and is applied to the patient and used in the same manner. The limb is prepared and the casting sleeve and elastic bands are applied in the same manner as shown in FIGS. 15 through 18 for the previous embodiments. Thereafter, first impermeable sheet 142 is placed on top of inelastic sheet 502. Second impermeable sheet 143 is placed on top of sleeve assembly 20. Inelastic sheet 502, having fastener pairs along its lateral edges and an axial length greater than the length of castable sleeve assembly 20 is positioned around the limb and the fastener pairs joined to form a tubular member having a diameter approximately equal to the inner diameter of the coil. Expandable member 504 is positioned on top of the limb, sleeve assembly 20, and impermeable sheets 142 and 143, and inelastic sheet 502, and the limb and sleeve assembly and sheets positioned in coil 506 by axial insertion. Expandable member 504 is inflated using inflation tube 505. Fill tubes 68 are connected via secondary fill tubes 162 to primary fill tube 164. Primary fill tube 164 is connected to the mixing nozzle of a foam chemical component supply system equipped with a valve, 166. Opening valve 166 for a predetermined length of time injects the proper amount of mixed components to space 40 (FIGS. 2 through 6) of sleeve assembly 20. Filling occurs in the same sequential manner as the previous embodiments, the flow of the expanding liquid/foam material being determined by elastic bands 144, 148, 152 and 156. Expansion of excess foam within sleeve assembly 20 outside proximal end 302 and distal end 304 of coil 506 is contained within the tubular structure formed by inelastic sheet 502. This confinement of the excess foams extends the length of the cast beyond the length of coil 506 providing additional immobilization of the limb confined therein. When the imaging procedure is complete, expandable member 504 is collapsed so as to provide clearance for removal of the cast from coil 506. The cast is removed axially from the coil. In this embodiment the collapsible member is an inflatable vessel. In other embodiments the expandable member is a mechanical assembly (as depicted in FIGS. 20-29).

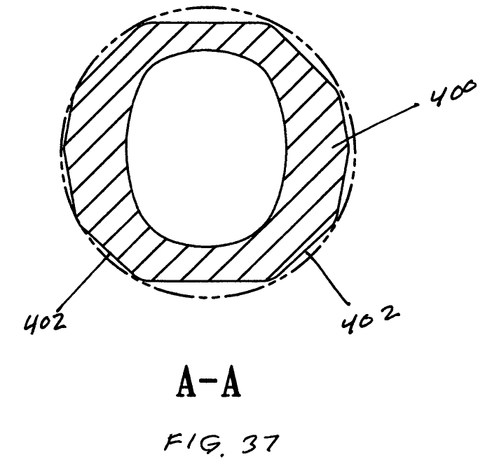

FIGS. 36 and 37 depict an embodiment for immobilizing a subject's head in the coil of an MRI. Cast 400 is formed in a mold in the same manner as the previous embodiments formed in a mold, but using a casting sleeve assembly configured to have a proximal end which surrounds the upper portion of the head and a distal portion having a closed end. As seen in FIG. 37, which depicts a section view at location A-A of FIG. 31, cast 400 has locating flats 402 which locate against the lower portions of the interior of an MRI coil. Cast 400 is immobilized within the coil by the expandable member (e.g., an inflatable vessel or other means described previously). In another embodiment, casting 400 is formed in place, in the coil in the manner of the embodiments of FIGS. 33 and 34. Cast 400 is removed from the body portion in the same manner as other embodiments described herein.

The disclosure of each publication, patent or patent application mentioned in this specification is specifically incorporated by reference herein in its entirety.

The invention has been illustrated by reference to specific examples and preferred embodiments. However, it should be understood that the invention is intended not to be limited by the foregoing description, but to be defined by the appended claims and their equivalents.

What is claimed:

1. A casting sleeve for forming a rigid cast for immobilizing a body part of a subject, said sleeve comprising:
(a) an upper proximal sheet and a lower distal sheet affixed to each other along their respective edges so as to define an enclosed space therebetween for insertion of an expanding polymeric foam material, said sheets being impermeable to said foam material;
(b) a first set of mating fastener pairs disposed at first and second lateral edges of said upper proximal sheet;
(c) a plurality of openings disposed in said lower distal sheet, providing access to said enclosed space, each of said openings having a fitting mounted therein for fluid tight connection to a fill tube that delivers said expanding foam material to said enclosed space;
wherein when said mating fastener pairs are connected, a double walled tubular sleeve having a projecting seam along its longitudinal axis is formed.

2. The casting sleeve of claim 1, further comprising a second set of mating fastener pairs disposed on said upper proximal sheet at a predetermined distance from said first set of mating fastener pairs.

3. The castable sleeve of claim 1, further comprising a plurality of elastic bands circumferentially disposed about said sleeve, both proximal and distal to said fill tube fittings, forming a series of communicating chambers along the length of said casting sleeve.

4. A rigid cast for immobilizing a body part of a subject, said cast formed by (a) wrapping the casting sleeve of claim 1 about a body part of a subject, such that said upper proximal sheet is facing said body part, (b) connecting the mating fastener pairs so as to form a double-walled tubular sleeve having a projecting seam along its longitudinal axis, (c) filling the enclosed space of said sleeve with an expandable polymeric foam material via fill tubes connected to said fill tube fittings and (d) allowing said foam material to solidify.

5. A rigid cast for immobilizing a body part of a subject, said cast formed by (a) wrapping the casting sleeve of claim 1 about a body part of a subject, such that said upper proximal sheet is facing said body part, (b) connecting the mating fastener pairs so as to form a double-walled tubular sleeve having a projecting seam along its longitudinal axis, (c) placing the casting sleeve within a mold assembly, (d) filling the enclosed space of said sleeve with an expandable polymeric foam material via fill tubes connected to said fill tube fittings and (e) allowing the foam material to solidify, wherein said mold assembly is shaped so as to provide the rigid cast with a cross-section that prevents rotation about the longitudinal axis of the cast when said cast disposed within an imaging coil assembly.

6. The rigid cast of claim 5, wherein said cross-section comprises an irregular polygon.

7. The rigid cast of claim 5, wherein said mold assembly comprises upper and lower portions, said upper portion including a plurality recesses that provide the upper surface of said rigid cast with a corresponding plurality of raised surfaces that prevent movement of the cast relative to an imaging coil assembly.

8. The rigid cast of claim 7, wherein said raised surfaces comprise ribs or ridges disposed along the length of the cast.

9. The rigid cast of claim 7, wherein said raised surfaces comprise protuberances or bumps disposed along the length of the cast.

10. The rigid cast of claim 5, wherein said mold assembly comprises upper and lower portions, each of which comprises of a series of axially spaced, connected plates.

11. An immobilizing assembly for stabilizing and restraining a body part of a subject in a coil of an imaging device comprising:
(a) the rigid cast of claim 4 disposed within said imaging coil;
(b) an expandable member disposed between the outer surface of said cast and the inner surface of said imaging coil, wherein said expandable member, when expanded, applies a unidirectional force perpendicular to one or more locating features on said cast so as to prevent movement of the cast relative to the imaging coil.

12. The immobilizing assembly of claim 11, wherein said expandable member comprises an inflatable bladder.

13. The immobilizing assembly of claim 11, wherein said expandable member comprises a resilient member selected from a compressible spring and elastomeric layer.

14. The immobilizing assembly of claim 11, wherein said expandable member has mechanically retracted and expanded configurations, such that when said expandable member is in the expanded configuration, it applies a unidirectional force to said cast that prevents movement of the cast relative to the imaging coil.

15. A method of immobilizing a body part of a subject during an imaging procedure comprising the steps of:
(a) inserting the rigid cast of claim 4 into an imaging coil assembly;
(b) providing an expandable member between the outer surface of said cast and the inner surface of said imaging coil assembly, said expandable member contacting the rigid cast only at the upper surface of said cast;
(c) expanding said expandable member such that it applies a unidirectional force perpendicular to one or more locating features on said cast so as to prevent movement of the cast relative to the imaging coil.

16. The method of claim 15, wherein said expandable member comprises an inflatable bladder and said expanding step comprises filling said bladder with a conformable substance.

17. The method of claim 16, wherein said conformable substance is a fluid.

18. The method of claim 15, wherein said expandable member comprises has mechanically retracted and expanded configurations, such that when said expandable member is in the expanded configuration, it applies a unidirectional force to said cast that prevents movement of the cast relative to the imaging coil.
